(19) United States
(12) Patent Application Publication
(10) Pub. No.: US 2012/0039967 A1
(43) Pub. Date: Feb. 16, 2012

(54) Low Rub-Off Compositions

(75) Inventors: Anjing Lou, Seymour, CT (US); Pravin Shah, Rutherford, NJ (US); Qiang Qiu, Trumbull, CT (US); Wei Dong Tian, Shelton, CT (US)

(73) Assignee: CONOPCO, INC., D/B/A UNILEVER, Englewood Cliffs, NJ (US)

(21) Appl. No.: 12/855,348

(22) Filed: Aug. 12, 2010

(51) Int. Cl. A61K 8/02; A61Q 1/02; A61K 8/92

(52) U.S. Cl. 424/401; 424/63

(57) ABSTRACT

Low rub-off compositions comprising a silicon-based particle that encapsulates a colorant are described. The compositions can comprise a color generating component and are suitable to immediately impart an even color to skin when topically applied.

### FIELD OF THE INVENTION

The present invention is directed to a low rub-off composition. More particularly, the invention is directed to a topical composition that is suitable to deliver a colorant to skin with or without color generating component. The composition comprises a silicone-based particle that encapsulates the colorant. When applied, the composition unexpectedly imparts an even color to skin, is low rub-off and easy to remove with soap and water.

### BACKGROUND OF THE INVENTION

Many dermatologists and consumers recognize the health concerns associated with sunbathing. In fact, it is widely accepted that “tan-happy” sunbathers are at risk for skin growths, including melanoma. Melanoma, if allowed to advance, can be lethal.

Notwithstanding the serious consequences that often result from sunbathing, many consumers enjoy looking tan. To avoid the ultraviolet rays of the sun, prudent consumers rely on sunless tanning compositions which effectuate a tan through cosmetic means. These compositions tend to employ dihydroxyacetone (DHA) to accomplish self-tanning of the skin. See J. Soc. Cosmet. Chem., 35, pages 265-272 (August 1984).

While sunless tanning compositions do exist, they often do not attract certain sunbathing loyalists because the same can impart uneven coloring (often with an orange tint). Moreover, compositions with colorants that yield immediate color benefits, including make-ups, often rub off onto clothing or other fabric-based objects like table cloths and couches.

In addition to consumers that enjoy the tan look, there exist consumers that prefer a lighter or white look. These consumers are good at avoiding the sun and they often use immediate optical benefit compositions to impart a very white skin complexion. While whitening compositions are available, the same have drawbacks (similar to those associated with sunless tanning compositions) in that they are not easy to apply evenly and they do have colorants that rub-off on clothing and other fabric-based objects.

There are increasing interests to develop compositions that provide immediate color benefits, evenly impart color, do not easily rub off when colorants are used and are easy to remove with soap and water. This invention, therefore, is directed to a low rub-off composition that is suitable to deliver a colorant to skin with or without color generating component. The composition comprises a silicone-based particle that encapsulates the colorant wherein the particle can also provide attractive visual cues within the composition which is often white or opaque in color. When applied, the composition unexpectedly imparts an even color to skin, is low rub-off and easy to remove with soap and water. Such a composition, surprisingly, imparts immediate color benefits while simultaneously being low rub-off.

### ADDITIONAL INFORMATION

Efforts have been disclosed for developing compositions that generate color. In U.S. Pat. No. 5,612,044, compositions with DHA and a silicone are described.

Other efforts have been disclosed for making compositions that generate color. In WO 97/33560, compositions with DHA in a multi-compartment dispenser are described.

Even other efforts have been disclosed for making compositions that generate color. In U.S. Patent Application No. 2009/0155321, compositions with encapsulated coloring agents are described.

None of the additional information above describes a composition with a silicone-based particle that encapsulates colorant as described in this invention.

### SUMMARY OF THE INVENTION

In a first aspect, the present invention is directed to a silicone-based particle that encapsulates colorant.

In a second aspect, the present invention is directed to a composition suitable to deliver a colorant to skin with or without color generating component, the composition comprising the particle of the first aspect of this invention.

In a third aspect, the present invention is directed to a method for coloring skin with the composition of the second aspect of this invention.

All other aspects of the present invention will more readily become apparent upon considering the detailed description and examples which follow.

Skin, as used herein, is meant to include skin on the face, neck, chest, back, arms, hands, legs, buttocks and scalp. Composition is meant to include a product ready for a consumer to topically apply (i.e., end use composition), including to topically apply to hair. Color generating component is meant to mean a component or additive that does more than physically color skin like, for example, DHA which colors skin via a biological mechanism or mechanisms and niacinamide which lightens skin via non-physical means. Colorant is meant to include a component like a dye and/or pigment that physically colors skin. The same is meant to include carotenoids such as lycopene. Colors skin, as used in this invention, means color modulation, and therefore, rendering skin darker or lighter. Encapsulated means coated with or embedded in. Viscosity, unless defined otherwise, means a fluids internal resistance to flow taken at a shear rate of 1 s⁻¹ at ambient temperature with a strain controlled parallel plate rheometer (made commercially available from suppliers like T.A. Instruments under the Ares name). Silicone-based means comprising a non-emulsifying elastomer (i.e., a siloxane where polyoxyalkylene units are absent). Low rub-off means having a Delta E* of less than 3.75 as defined in Example 2. Comprising, as used herein, is meant to include consisting essentially of and consisting of: All ranges defined herein are meant to include all ranges subsumed therein unless specifically stated otherwise.

### DETAILED DESCRIPTION OF THE PREFERRED EMBODIMENTS

The only limitation with respect to the type of non-emulsifying elastomer that may be used in this invention is that the same is suitable for use in a topical composition. Illustrative yet non-limiting examples of the types of non-emulsifying elastomers that may be used in this invention include those that have an average number (Mn) molecular weight in excess of 2,000, and preferably, in excess of 5,000, and most preferably, in the range from about 10,000 to about 20 million, including all ranges subsumed therein. Often, the elastomers are formed from a divinyl compound which has at least two free vinyl groups, reacting with Si—H linkages of a polysiloxane backbone. Such elastomer compositions are commercially available under the proposed CTFA name of Cyclomethicone and Vinyl Dimethicone Methicone Cross Polymer, delivered as 20-35% elastomer in a cyclomethicone carrier. A related elastomer composition under the CTFA name of Crosslinked Stearyl Methyl Dimethyl Siloxane Copolymer is available as Gransil SR-CYC (25-35% elastomer in a cyclomethicone carrier) from Grant Industries, Inc., Elmwood Park, N.J. The commercial products are typically further processed by subjecting them to a high pressure (approximately 5,000 psi) treatment in a Sonolator with recycling in 10 to 60 passes. Sonolation achieves a resultant fluid with elastomer average particle size ranging from 0.2 to 10 micron, preferably 0.5 to 5 micron. Viscosity is preferred often when ranging between 300 and 20,000 cps at 25° C., as measured by a Brookfield LV Viscometer (size 4 bar, 60 rpm, 15 sec). In an especially preferred embodiment, the most desired non-emulsifying elastomers are cyclomethicone/dimethicone cross-polymers made commercially available by suppliers like Dow Chemical under the names DC9040 and DC9045, and Shin-Etsu under the name KSG-15 elastomer (with about 6-13% by weight cross-linked polymer in a cyclomethicone carrier).

The particle comprising colorant typically comprises from about 60 to about 95%, and preferably, from about 65 to about 90%, and most preferably, from about 70 to about 85% by weight non-emulsifying elastomer (including carrier) based on total weight of particle and including all ranges subsumed therein.

In addition to non-emulsifying elastomer, optional but often preferred oils (including petroleum jelly) may be used along with the non-emulsifying elastomer to generate particle, as long as the same do not interfere with the encapsulation of colorant. Excluding petroleum jelly, such oils typically have a viscosity of less than about 200 centipoise, and preferably, less than about 150 centipoise, and most preferably, from about 5 to about 120 centipoise, including all ranges subsumed therein.

Illustrative yet non-limiting examples of specific optional oils suitable for use in this invention include hydrocarbon-based oils such as mineral oil and oils having 8-16 carbon atoms like branched C₈-C₁₆ alkanes. Suitable branched alkanes include isoparrafins like, for example, isodecane, isododecane, isohexadecane and mixtures thereof.

Other oils that may be used with non-emulsifying elastomer to generate particle and encapsulate colorant include silicones (linear and/or cyclic) and especially those comprising from about 2 to about 12 silicon atoms whereby such silicones comprising alkyl or alkoxy groups have from about 1 to about 22 carbon atoms. Often preferred oils include decamethyl cyclopentasiloxane, octamethyl cyclotetrasiloxane, dodecamethyl cyclohexasiloxane, heptamethyl hexyltrisiloxane, heptamethyloctyl trisiloxane, heptamethyloctyl trisiloxane, hexamethyl disiloxane, octamethyl trisiloxane, decamethyl tetrasiloxane, dodecamethyl pentasiloxane, mixtures thereof or the like. In an often preferred embodiment, the optional oil used is decamethyl cyclopentasiloxane, made commercially available by Dow Chemical under the DC245 name. Still other oils suitable for use to form particle and encapsulate colorant include non-volatile oils like germ oil, sunflower oil, castor oil, sesame seed oil, cottonseed oil, safflower oil, soybean oil, maize oil, pumpkin oil, mixtures thereof or the like.

In an especially preferred embodiment, oil is used with the non-emulsifying elastomer to generate the particle to encapsulate colorant where the particle often comprises from about 0.1 to about 50%, and preferably, from about 2 to about 45%, and most preferably, from about 5 to about 40% oil by weight, based on total weight of non-emulsifying elastomer and oil used to generate the particle and including all ranges subsumed therein.

The particle comprising colorant typically comprise from about 5 to about 40%, and preferably, from about 10 to about 35%, and most preferably, from about 15 to about 30% by weight colorant, based on total weight of particle and including all ranges subsumed therein.

The particle with colorant can be made by mixing colorant with non-emulsifying elastomer (and optionally oil) to generate the desired particles. Moderate shear should be employed and the mixing temperature should range from about ambient temperature to about 55° C. The resulting particles, after being distributed in end use composition, typically have a diameter from about 0.01 to about 3 mm, and preferably, from about 0.05 to about 2.5 mm, and most preferably, from about 0.1 to about 2 mm, including all ranges subsumed therein. To the extent the particles are not a perfect sphere, the diameter is meant to be taken at the widest portion of the particle. Often, from about 25 to about 75%, and preferably, from about 30 to about 65%, and most preferably, from about 40 to about 60% of the diameter of the particle (taken as a cross-section of particle) is non-emulsifying elastomer or non-emulsifying elastomer and oil encapsulant as opposed to colorant center or core. The non-emulsifying elastomer or non-emulsifying elastomer and oil typically is/are about homogeneously/evenly applied around the colorant where about homogeneously is meant to mean the thickest and thinnest portion are within about 30%, and preferably, within about 25% of each other.

In a preferred embodiment, storage modulus, G′, for the silicone-based particle comprising colorant of this invention is from about 3 to about 12, and preferably, from about 4 to about 10 times higher than its loss modulus, G″when taken at about 10% strain at ambient temperature and with a parallel plate rheometer like one sold under the Ares name.

Illustrative dye colorants suitable for use in this invention include FD&C Yellow 5, FD&C Yellow 6, D&C Yellow 10, D&C Red 6, D&C Red 7, D&C Red 21, D&C Red 27, D&C Red 28, D&C Red 30, D&C Red 36, D&C Red 40, D&C Green 6, carmine, D&C blue 1, F D&C blue 1, bromo dyes, fluoroescein dyes, or mixtures thereof.

Suitable pigments for use as colorant in this invention include but are not limited to, titanium dioxide, calcium carbonate, clay, talc, barium sulfate, white carbon, bismuth oxychloride, chromium oxide, zinc oxide, zinc sulfide, zinc powder, metal oxide coated mica (such as titanium oxide coated mica), thin platelet-like alumina, metal oxide coated thin platelet-like alumina (such as titanium dioxide coated thin platelet-like alumina), iron oxide, magnesium carbonate, hydroxyapatite or mixtures thereof.

Preferred colorants suitable for use include carotenoids, chromium oxides, iron oxides, titanium oxides, cobalt salts, charcoal and mixtures thereof. Most preferred is iron oxide. In an especially preferred embodiment, iron oxide of red, yellow and/or black color is used. For these embodiments the relative weight ratio of black to red may range from 2:1 to 1:2, preferably from 1.5:1 to 1:1. The weight ratio of black to yellow may range from 6:1 to 1:1, preferably from 4:1 to 2:1, optimally from 3:1 to 2.2:1.

When making desired low rub-off compositions suitable to deliver colorant, the same typically comprise from about 0.1 to about 6%, and preferably, from about 0.2 to 5%, and most preferably, from about 0.4 to about 4% by weight particle, based on total weight of the composition and including all ranges subsumed therein.

Color generating component suitable for use in this invention is limited only to the extent that the same may be formulated in a topical composition used by consumers. Illustrative yet non-limiting examples of the types of color generating component that may be used in this invention include those generally classified as alpha-hydroxyaldelydes and ketones, glyceraldehyde, troxerutin and related alcohol aldehydes, various indoles, imidazoles and derivatives thereof.

Suitable color generating components that may be used often include DHA, melanin, mahakanni (eclipta alba), methyl glyoxal, erythrulose, alloxan, 2,3-dihydroxysuccindialdehyde, mixtures thereof, or the like.

Additional suitable color generating components suitable for use include skin lightening agents such as vitamin C, and vitamin B₃ (niacinamide), kojic acid, ferulic acid, resorcinols like 4-substituted resorcinols, as well as extracts like placental extract, yarrow extract, chamomile extract, arbutin (i.e., beanberry plant extract) or mixtures thereof.

When used, the color generating components that are classified as sunless tanning agents typically make up from about 0.025 to about 35%, and preferably, from about 0.05 to about 15%, and most preferably, from about 0.5 to about 10% by weight of the composition suitable to deliver a colorant to skin, based on total weight of the composition and including all ranges subsumed therein.

Such skin lightening agents, when used, typically make up from about 0.002 to about 12%, and preferably, from about 0.1 to about 10%, and most preferably, from about 0.5 to about 6% by weight of the composition suitable to deliver colorant to skin, based on total weight of the composition and including all ranges subsumed therein.

In an often preferred embodiment, the relationship between the colorant and the color generating component is one which is symbiotic. This means, for example, that the silicone-based particle that encapsulates colorant is typically designed to instantly deliver color that can enhance the effect of the color generating component, whereby enhancing includes either physically delivering color quickly that is substantially the same as the color of skin treated with color generating component and/or that renders any negative results (e.g., unevenness and/or off colors like orange tints) obtained when using the color generating component less visible or even not noticeable. Moreover, the composition comprising silicone-based particle that encapsulates colorant is typically applied to skin with shear to allow for release of colorant and spreading of the non-emulsifying elastomer or non-emulsifying elastomer and oil encapsulant as a film that prevents the rubbing off of colorant from skin to another object like fabric.

In an especially preferred embodiment, the color generating component used in this invention is a self-tanning agent like DHA and the colorant is an iron oxide, and preferably, an iron oxide mixture of red, yellow and black color.

Composition suitable to deliver particle that encapsulates colorant of the present invention is not typically limited by any pH range. However, a preferred pH ranges from about 3.0 to about 7, and preferably, from about 3.5 to about 6, and most preferably, from about 4 to about 5.5. The same may be emulsions which are water-in-oil or oil-in-water where oil-in-water emulsions are often preferred. Emulsifier typically makes up from about 0.25 to about 12% by weight of the composition. Suitable emulsifiers often include glyceryl stearate, PEG-100 stearate, glycol stearate, cetyl alcohol or mixtures thereof. A desired composition typically has a viscosity from about 3,000 to about 65,000 centipoise, and preferably, from about 4,000 to about 40,000 centipoise, and most preferably, from about 5,000 to about 35,000 centipoise, including all ranges subsumed therein.

Thickeners may be present. Illustrative thickeners are the chemically modified starches such as sodium hydroxypropyl starch phosphate available from the National Starch and Chemical Company and from Grain Processing Corporation, the latter under the trademark Pure-Gel®. Tapioca starch is also an often preferred thickener. Polymers such as taurates may be useful as thickeners. One example is hydroxyethyl acrylate/sodium acryloyldimethyl taurate copolymer. Usually from about 0.1 to about 5% by weight thickener is used.

A variety of other components may be present in the compositions of the present invention. Foremost is that of water. Amounts of water may range from about 1 to about 90%, preferably from about 30 to about 80%, optimally from about 40 to about 70% by weight.

The compositions of the present invention may include glycerin for moisturization. Amounts of glycerin when present may range from about 1% to about 25%, preferably from 5% to 20%, more preferably from 8 to 15%, optimally from 10 to 15% by weight of the composition.

Emollient materials may be included as carriers in compositions of this invention. These may be in the form of silicone oils, synthetic esters and hydrocarbons. Amounts of the emollients may range anywhere from about 0.1 to about 95%, preferably between about 1 and about 50% by weight.

Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicon atoms.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes with kinematic viscosities of from about 5×10⁻⁶ to 0.1 m²/s at 25° C. Among the preferred nonvolatile emollients useful in the present compositions are the polydimethyl siloxanes having viscosities from about 1×10⁻⁵ to about 4×10⁻⁴ m²/s at 25° C.

Another class of nonvolatile silicones suitable for use as carrier are the non-emulsifying silicone elastomers used to generate the silicon-based particle that encapsulates colorant.

Among the ester emollients suitable for use are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols.
(4) Wax esters such as beeswax, spermaceti wax and tribehenin wax.
(5) Sterols esters, of which cholesterol fatty acid esters are examples thereof.
(6) Sugar ester of fatty acids such as sucrose polybehenate and sucrose polycottonseedate.

Hydrocarbons are suitable further ingredients in the composition comprising the silicon-based particle that encapsulates colorant. These include petrolatum, mineral oil, C₁₁-C₁₃ isoparaffins, polyalphaolefins, and especially isohexadecane, available commercially as Permethyl 101A from Presperse Inc.

Humectants of the polyhydric alcohol-type in addition to glycerin can be employed with the formulations of this invention. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of humectant may range anywhere from 0.5 to 50%, preferably between 1 and 15% by weight of the composition.

Sunscreen actives may also be included in compositions of the present invention. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide, zinc oxide, polyethylene and various other polymers. Amounts of the sunscreen agents when present may generally range from 0.1 to 30%, preferably from 2 to 20%, optimally from 4 to 10% by weight.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from 0.01% to 2% by weight of the composition.

Compositions of the present invention may also contain optional water-soluble vitamins like Vitamin B₂, Vitamin B₆, and Biotin. Among the useful water-insoluble vitamins are Vitamin A (retinol), Vitamin A Palmitate, Ascorbyl Tetraisopalmitate, Vitamin E (tocopherol), Vitamin E Acetate and DL-panthenol. Anti-Aging actives may also be present including flavanoids, resveratrol and epidihydroandrosterol (DHEA). Total amount of vitamins and anti-aging actives when present in compositions according to the present invention may range from 0.001 to 10%, preferably from 0.01% to 1%, optimally from 0.1 to 0.5% by weight.

Desquamation agents are further optional components. Illustrative are the alpha-hydroxycarboxylic acids and beta-hydroxycarboxylic acids. Among the former are salts of glycolic acid, lactic acid and malic acid. Salicylic acid is representative of the beta-hydroxycarboxylic acids. Chelators like EDTA are also suitable for use as are pH buffers like citric acid. Materials comprising hydrogenated olive oils, like Oliwax LC may also be used to stabilize the compositions. Amounts of these types of materials when present may range from about 0.1 to about 15% by weight of the composition.

Basic colors (i.e., colors added to carrier), fragrances, opaciflers and abrasives may also be included in compositions of the present invention. Each of these substances may range from about 0.05 to about 5%, preferably between 0.1 and 3% by weight.

Packaging for the composition described herein is not limited and often is a bottle, tube roll-ball applicator, squeeze container or lidded jar.

The following examples will more fully illustrate the embodiments of this invention. Such examples are not intended to limit the scope of the claims.

### Examples 1

| | | Com- | Com- | | |
|---|---|---|---|---|---|
| | | parative | parative | Sample | Sample |
| Phase A | Ingredient Name | Sample 1 | Sample 2 | 3 | 4 |
| Phase | Deionized water | Balance | Balance | Balance | Balance |
| | Thickener | 1.00 | 1.00 | 1.00 | 1.00 |
| | Opacifier | 0.50 | 0.50 | 0.50 | 0.50 |
| | Glycerin | 12.0 | 0.00 | 0.00 | 0.00 |
| | Propylene glycol | 0.00 | 12.00 | 12.00 | 12.00 |
| | Preservative | 0.70 | 0.70 | 0.70 | 0.70 |
| | Disodium EDTA | 0.05 | 0.05 | 0.05 | 0.05 |
| | Citric acid | 0.02 | 0.02 | 0.02 | 0.02 |
| | Glyceryl stearate | 2.40 | 2.40 | 2.40 | 2.40 |
| | Cetyl alcohol | 2.40 | 2.40 | 2.40 | 2.40 |
| | PEG-100 stearate | 1.20 | 1.20 | 1.20 | 1.20 |
| | Dimethicone 50 cst | 3.00 | 3.00 | 3.00 | 3.00 |
| | Isopropyl palmitate | 2.00 | 2.00 | 2.00 | 2.00 |
| | Oliwax LC | 1.00 | 1.00 | 1.00 | 1.00 |
| | Tapioca starch | 0.50 | 0.50 | 0.50 | 0.50 |
| | DHA | 1.50 | 1.50 | 1.50 | 1.50 |
| | Base Color | 0.35 | 0.35 | 0.35 | 0.35 |
| Phase B | Oil Pigment phase | | | | |
| | Petrolatum | 2.50 | 2.50 | — | — |
| | DC 9045 | 0.00 | 0.00 | 2.00 | 1.65 |
| | DC 245 | 0.00 | 0.00 | 0.50 | 0.85 |
| | Iron oxide red | 0.25 | 0.25 | 0.25 | 0.25 |
| | Iron oxide yellow | 0.10 | 0.10 | 0.10 | 0.10 |
| | Iron oxide black | 0.25 | 0.25 | 0.25 | 0.25 |

The ingredients of Phase A (deplete of fragrance and DHA) were mixed and stirred under moderate shear. Heat was supplied to yield a mixture having a temperature of about 70° C. The resulting mixture was subjected to sonification for about one minute and cooled to about 35° C. Moderate shear was again used in order to blend in fragrance and DHA.

In phase B, colorant was dispersed in either Petrolatum (Comparative Samples 1 and 2) or in non-emulsifying elastomer and oil (Samples 3 and 4). Ingredients were mixed under moderate shear until all colorant was dispersed uniformly. Particles in the comparative samples, made with Petrolatum as the encapsulant, were heated to about 50° C. during manufacture.

The phases were mixed to yield end use compositions with particles encapsulating colorant. Mixing was achieved with moderate shear and was complete when particles with colorant were evenly distributed, having diameters of about 2 mm.

### Example 2

Samples prepared in Example 1 were applied to artificial skin substrates (23 cm×7 cm) made commercially available by Kittrich. Each sample was applied to artificial skin where a film applicator (1μ) was run over the sample to make a 25 μm film of sample on artificial skin. The artificial skin had about 2 mg of sample per cm² of skin. White discs of cotton fabric (3.5 cm² radius, 2 mm thick) were placed on plastic discs (radius about 2.7 cm).

The discs, cloth down, were placed on top of artificial skin with sample and controlled loading pressure (30 g/cm²) applied over the disc surface. The discs were moved by a drawing force on the surface of the artificial skin at a rate of 2.5 cm/s. Temperature was circa 21° C. and the relative humidity was about 25 to 40%.

The discs and cloths were removed from the artificial skin having sample applied thereon. The white cloths were assessed for color change utilizing a commercially available colorimeter. Color change, (Delta E*), of the fabric before and after contact with the artificial skin is described as the square root of (delta L*)²+(delta a*)²+(delta b*)². Five measurements were taken at different spots of the fabric to get the average values in the table below.

| | TABLE |
|---|---|
| | Delta E* |
| Comparative Sample 1 | 5.4 |
| Comparative Sample 2 | 4.0 |
| Sample 3 | 3.5 |
| Sample 4 | 3.1 |

The results unexpectedly demonstrate that compositions made consistent with this invention are less likely to rub off than conventionally made comparative samples having petrolatum encapsulated colorant.

The artificial skin having sample applied thereon was assessed visually for unevenness and washability. A unevenness scale ranging from 1 to 5 was applied with 5 being the most uneven and 1 being the most even. Unexpectedly, the artificial skin substrates subjected to sample compositions made consistent with this invention scored a 2 after visual assessment whereby the comparative samples scored a 5.

Soap, like Dove®, and water was used to wash/remove the composition of this invention after application to skin. The composition of this invention was, unexpectedly, easy to remove notwithstanding the excellent low rub-off properties.

What is claimed is:

1. A silicon-based particle comprising encapsulated colorant, the silicon-based particle comprising from about 60 to about 95% by weight non-emulsifying elastomer.

2. The silicon-based particle according to claim 1 wherein the colorant is a dye, pigment or mixture thereof.

3. The silicon-based particle according to claim 1 wherein oil is combined with non-emulsifying elastomer.

4. The silicon-based particle according to claim 3 wherein the oil comprises siloxane.

5. The silicon-based particle according to claim 1 where the non-emulsifying elastomer comprises cyclomethicone/dimethicone cross-polymer.

6. A composition for delivering colorant to skin, the composition comprising:
(a) a silicon-based particle comprising encapsulated colorant, the silicon-based particle comprising from about 60 to about 95% by weight non-emulsifying elastomer; and
(b) carrier.

7. The composition for delivering colorant to skin according to claim 6 wherein the composition further comprises a color generating component and the colorant is a dye, pigment or mixture thereof.

8. The composition for delivering colorant to skin according to claim 7 wherein the color generating component is a sunless tanning agent or skin lightening agent and the silicon-based particle comprising encapsulated colorant has a diameter from about 0.01 to about 3 mm.

9. The composition for delivering colorant to skin according to claim 8 wherein the color generating component is a sunless tanning agent and the sunless tanning agent is DHA, melanin, mahakanni, methylglyoxal, erythrulose, alloxan, 2,3-dihydroxysuccindialdehyde or a mixture thereof.

10. The composition for delivering colorant to skin according to claim 9 wherein the sunless tanning agent is DHA.

11. The composition for delivering colorant to skin according to claim 9 wherein the colorant yields an immediate color to skin that is substantially the same as color of skin having been treated with the sunless tanning agent.

12. The composition for delivering colorant to skin according to claim 11 wherein the colorant is an iron oxide or mixture of iron oxides.

13. The composition for delivering colorant to skin according to claim 8 where the color generating component is a skin lightening agent and the skin lightening agent is Vitamin C, Vitamin B₃, Kojic acid, ferulic acid or a resorcinol.

14. The composition for delivering colorant to skin according to claim 13 wherein the skin lightening agent is Vitamin B₃.

15. The composition for delivering colorant to skin according to claim 13 wherein the colorant yields an immediate color to skin that is substantially the same as color of skin having been treated with the skin lightening agent.

16. The composition for delivering colorant to skin according to claim 6 where the silicon-based particle comprising encapsulated colorant yields a low rub-off composition after application to skin.

17. The composition for delivering colorant to skin according to claim 6 wherein the composition comprises from about 0.1 to about 6% by weight particle.

18. The composition for delivering colorant to skin according to claim 6 wherein the carrier comprises water and oil and is an emulsion.

19. A method for immediately coloring skin with a low rub-off composition, the method comprising the step of topically applying to skin the composition of claim 6.
